(19) United States
(12) Patent Application Publication
(10) Pub. No.: US 2018/0093029 A1
(43) Pub. Date: Apr. 5, 2018

(54) Balancing Unit Being Independent of Temperature Disturbance and Balancing Method

(71) Applicant: Fresenius Medical Care Deutschland GmbH, Bad Homburg (DE)

(72) Inventors: Petra Abel, Friedberg (DE); Alexander Heide, Eppstein (DE); Dejan Nikolic, Frankfurt (DE); Arne Peters, Bad Homburg (DE); Elke Schulte, Schweinfurt (DE); Christoph Wiktor, Gelnhausen (DE)

(21) Appl. No.: 15/559,544

(22) PCT Filed: Mar. 21, 2016
(86) PCT No.: PCT/EP2016/056151

§ 371 Date: Sep. 19, 2017

(30) Foreign Application Priority Data
Mar. 24, 2015 (DE) 10 2015 104 430.7

(51) Int. Cl. A61M 1/16

(52) U.S. Cl. CPC A61M 1/1635; A61M 1/1647; A61M 1/1662; A61M 2205/3368; A61M 2205/127; A61M 2205/3334; A61M 2205/366; A61M 1/1664

(57) ABSTRACT

The disclosure relates to a functional device, in particular a medical functional device, having at least one fluid circuit. The fluid circuit comprises at least one heat exchange device being arranged and/or embodied between a first section of the fluid circuit and a second section of the fluid circuit. The disclosure relates further to a balancing unit, a treatment apparatus, and a balancing method.

### CROSS REFERENCES TO RELATED APPLICATIONS

The present application is a national stage entry of International Patent Application No. PCT/EP2016/056151, filed on Mar. 21, 2016, and claims priority to Application No. 10 2015 104 430.7, filed in the Federal Republic of Germany on Mar. 24, 2015. The disclosures of the prior applications are expressly incorporated herein in entirety by reference thereto.

### TECHNICAL FIELD

This disclosure relates to a medical functional device, a balancing unit, a medical treatment apparatus, and a method for balancing fluid flows and/or for controlling or regulating at least one volume flow.

### BACKGROUND

In medicine, in particular, in the field of blood treatment, for example of dialysis, the patient is supplied with fluids and/or fluids are withdrawn from the patient. The exact balancing of the quantity of the fluids supplied and withdrawn is of importance for the safety and health of the patient.

Balancing units are used in practice to achieve the balance. They may be connected to blood treatment apparatuses, for example dialysis apparatuses.

During an extracorporeal blood treatment, heater or heating units are regularly used, for example to compensate for heat losses of blood outside the body. This may cause a complex temperature evolution of the liquids used in particular in the extracorporeal blood circuit, and also in the remaining fluid circuit. Thus, the fluids used or supplied for the extracorporeal circuit(s) may have different temperatures from each other. Due to the fact that temperatures may have an influence on the volume of the fluid(s), the temperature may influence not only the volume, but also the balancing. Stated another way, the balancing may be inaccurate when the influence of the temperature on the volume is not considered.

### SUMMARY

In one aspect, this disclosure describes balancing units and methods for balancing flows of fluid. In addition, medical functional devices as well as medical treatment apparatuses are described herein.

All or some of the advantages achievable by the functional devices described herein can be undiminishedly achieved also with the methods described herein, with the balancing units described herein and with the medical treatment apparatuses.

In another aspect, this disclosure describes a functional device, which is preferably a medical functional device. It comprises at least one fluid circuit, or at least one section thereof, or is connected thereto.

In addition, in some implementations the functional device and/or the fluid circuit comprise(s) or are/is connected with at least one heat exchange device. The heat exchange device is thereby at least arranged and/or embodied between two different sections of the fluid circuit, in particular between the first section and the second section.

In some implementations, the balancing units described herein are provided and embodied and/or configured to determine at least one fluid balance, in particular between at least one (or exactly one) first fluid flow in a first section of a fluid circuit and at least one-second volume flow in a second section of the fluid circuit. The balancing unit comprises thereby a functional device and/or it may be in data communication with at least one part of the functional device, in particular with at least one flow sensor and/or at least one pump, for example a blood pump, and/or is connected and/or connectable to at least one part of the functional device.

The medical treatment apparatus as described herein, which is embodied and/or configured for treating a medical fluid, comprises at least one balancing unit, and/or at least one functional device, and/or is connected to at least one of them, preferably in a detachable or releasable manner.

The method as described herein relates to the determination of at least one fluid balance between at least one first volume flow, in particular of one first section of a fluid circuit, and at least one second volume flow, in particular of a second section of the fluid circuit.

In some implementations, the method relates additionally or alternatively to the control and/or regulation (for better readability, both terms are referred to herein as “controlling”, which however optionally also encompasses regulating) of at least one volume flow of at least one section of the fluid circuit of the functional device. The method may also include the step of providing a functional device as described herein and/or a balancing unit as described herein.

In some implementations, the method encompasses at least the determination of at least one volume flow, for example in a first or in a second section.

In some implementations of the method, at least one fluid balance is formed from a first and a second volume flow.

In some implementations of the method, at least one fluid balance (here abbreviated as FB) is used for controlling and/or regulating at least one volume flow of the fluid circuit, a pump rate of at least one pump of the fluid circuit, and/or a cross section of at least one section of the fluid circuit.

Some embodiments may comprise some or all of the following features in any combination.

In all of the previous and following embodiments, the use of the expression “may be” or “may have” and so on, is to be understood synonymously with “preferably is” or “preferably has,” respectively, and so on, and is intended to illustrate embodiments.

Some embodiments may comprise some or all of the following features in any combination, provided this is not evidently technically impossible to those skilled in the art.

Whenever a numerical value is mentioned herein, the person skilled in the art understands it as an indication of a numerical lower limit. Unless it leads the person skilled in the art to an evident contradiction, the skilled person implicitly understands when specifying for example “one” always as “at least one”. This understanding is also evenhandedly encompassed in some embodiments, as the interpretation that a numeric word, for example, “one” may alternatively mean “exactly one”, wherever this is not evidently technically impossible for the skilled person. Both may be encompassed in certain aspects and apply herein to all used numerical values.

The spatial indications made herein such as “top”, “bottom”, etc. refer in case of doubt to the illustrations as can be seen in the accompanying figures.

Whenever the expressions “suitable”, “provided”, “designed”, “configured” and/or “programmed” is used herein, a person skilled in the art may understand a particular embodiment of the respective apparatus. The aforementioned expressions may be used herein interchangeably.

Although the present invention is described hereinafter primarily with reference to a dialysis treatment, the present invention is not limited thereto.

The expression “volume flow”, as used herein, may be understood to refer to a volumetric flow rate. That is, “volume flow” may be understood as the volume of a fluid which has moved or streamed or flowed within a given period of time through a cross-section (e.g., a cross-section of a portion of the fluid circuit) with respect to the corresponding given period of time. A volume flow may be exemplarily specified in ml per minute or per hour.

The expression “mass flow”, as used herein, may be equal to the density of the fluid multiplied by the volume flow.

The expression “determining”, as used herein, includes actions such as, but not limited to, adjusting, setting, estimating, obtaining, detecting, assuming, measuring, identifying, and reading or capturing (for example of a stored and/or set or adjusted value and/or a calculation, in particular from known, stored, determined and/or preset or adjusted values).

In certain exemplary embodiment, a “section of the fluid circuit” may be understood to be in particular the first section or the second section of the fluid circuit, in particular of a dialysate and/or substituate circuit.

A “volume flow of the fluid circuit” may be understood, in certain exemplary embodiments, to be in particular the volume flow of the first section or the volume flow of the second section of the fluid circuit, in particular of a dialysate and/or a substituate circuit.

In some exemplary embodiments, the in particular medical or medical technical functional device comprises at least one fluid circuit, in particular a process fluid circuit, for example a liquid circuit, or sections thereof.

The expression “fluid circuit”, as used herein, denotes a fluid system, a tube set, a blood tubing set or the like, each suitable, intended and/or embodied to receive, and be flowed through by, process fluids, medical liquids, e.g. blood, substituate, dialysate and/or combinations thereof. The process fluids may be used for the purpose of a process such as flushing, priming, substituting, decreasing or lowering the concentration of pollutants and the like. A fluid circuit must not allow or enable a closed circulation. A fluid circuit may comprise at least one filter device (e.g., a blood filter), and/or at least one pump (e.g., a blood or substituate pump).

In some exemplary embodiments, the fluid circuit or at least a section thereof is provided and/or designed to receive and/or to convey at least one fluid, in particular a medical fluid.

In some exemplary embodiments, the functional device comprises at least one channel which is at least suitable and/or embodied to convey and/or to lead a liquid.

In some exemplary embodiments, the fluid circuit is, in particular an arterial and/or venous, blood, dialysate and/or substituate circuit.

The functional device may further comprise at least one filter device, or at least one section thereof, which is arranged inside the fluid circuit, in particular between a blood circuit and at least one dialysate circuit. The filter device can be a blood filter or a dialyzer.

In some exemplary embodiments, the heat exchange device is dimensioned, arranged, connected and/or embodied such that the temperature of the first section and/or in particular the temperature of the fluid flowing through the first section of the fluid circuit and the temperature of the second section, in particular the temperature of the fluid flowing through the second section of the fluid circuit, are matched, balanced or substantially equal or identical within a pre-determinable, predefined, adjustable and/or pre-definable, at least limited or pre-determined time.

Alternatively or additionally, in some exemplary embodiments, the temperature difference of the fluids flowing through the respective sections and/or of the respective sections, is at least reduced, for example by about at least 10%, 20%, 30%, 40%, 50%, 60% or more before and after leaving the heat exchange device.

With the expression “balancing, balanced or equal”, as used herein, it may generally be understood that the temperatures of the relevant sections and/or of the fluids present in or flowing through the relevant sections after leaving the heat exchange device are equal or substantially equal. Thereby, particularly the temperature difference of the fluids flowing through the respective sections before and after leaving the heat exchange device may be reduced and/or balanced. In particular, the difference of the outgoing temperatures (of fluids flowing out of the heat exchange device) may be lower, for example significantly lower, than the difference of the incoming or initial temperatures.

Temperatures, having for example a temperature difference of less than or equal to 10° C. may be considered as “substantially” equal. Temperature differences of 2° C. or less, in particular of significantly lower than 1° C., are preferably achievable. The person skilled in the art will recognize that this specification may depend on the maximum temperature difference in the balancing circuit. Preferably, the maximum temperature difference should be reduced to about 30% to 70%.

In some exemplary embodiments, the first section is located upstream of a filter device, for example a blood filter, in an operating state of the functional device, in particular with respect to a flow direction of the fluid during the operation of the functional device, for example during a treatment. Alternatively or additionally, the second section may be located downstream of a filter device, for example a blood filter, in an operating state of the functional device, in particular with respect to a flow direction of the fluid during the operation of the functional device, for example during a treatment.

In certain exemplary embodiments, the heat exchange device is connectable, connected, and/or arranged at least with the first and/or at least with the second section of the fluid circuit (e.g., in a detachable, substitutable, or interchangeable manner).

In some exemplary embodiments, at least one section of the heat exchange device is integrally embodied or formed with at least one section of the fluid circuit.

In some exemplary embodiments, the heat exchange device is arranged and/or embodied in at least one section of the fluid circuit before at least one flow sensor in the fluid circuit (i.e., upstream to the latter), with respect to the flow direction of the fluid in a direction through the heat exchange device.

In some exemplary embodiments, the fluid circuit and/or the heat exchange device comprise(s) at least one heater.

In certain exemplary embodiments, the heater is arranged before, at, or after the heat exchange device, a flow sensor, or a filter device (with respect to the flow direction of the fluid during operation).

In some exemplary embodiments, the heat exchange device comprises at least one massive body, for example a hart part.

In some exemplary embodiments, at least one channel, at least one section of the fluid circuit, and/or at least one section, in particular a measuring section, of a flow sensor extend(s) into the massive body.

In some exemplary embodiments, the heat exchange device, or at least one part thereof, comprises at least one type of heat-conductive material (for example metal or metal alloy as well as ceramic, ceramic-filled plastic or plastic with thin walls), or it consists of one such material or a combination of such materials.

In some exemplary embodiments, at least one section of at least one flow sensor and/or at least one section of a fluid circuit, in particular a first and/or a second section, for example a measuring section of a flow sensor extends through at least one section of the heat-conducting body of the heat exchange device or is provided herein.

In some exemplary embodiments, at least one section of a flow sensor and/or at least one section of a fluid circuit is embodied in the functional device and/or in at least a heat-conducting body thereof as an inserted channel, preferably as a closed channel in cross section or as a non-closed channel, like a half-channel or part-channel.

In some exemplary embodiments, at least one channel or part-channel may be sealed against an outside by means of a layer, in particular a heat-conductive and/or fluid-tight layer (for example a film and/or a thin plate), and/or a further channel.

In some exemplary embodiments, two, in particular heat-conducting bodies (for example hard parts) may be connected or connectable to each other, preferably by at least one heat-conductive means (for example a heat-conducting paste and/or plate, such that the heat-conducting bodies are separable from each other).

In some exemplary embodiments, the heat exchange device is embodied as separate, detachable, interchangeable, and/or replaceable device, and/or as a single-use item or disposable (for example as a cassette, or as part thereof).

In certain embodiments, the functional device further comprises at least one flow sensor. Alternatively or additionally, it is configured and/or embodied to be connected and/or connectable with a flow sensor, in particular in a detachable manner.

In some exemplary embodiments, the flow sensor is embodied and/or arranged for measuring at least one volume flow in at least one section, such as in one first and/or in one-second section of the fluid circuit.

In some exemplary embodiments, the functional device and/or at least one section of the fluid circuit are/is intended and/or embodied to be connectable and/or connected and/or to be connected with at least one section of at least one flow sensor. Such a section can be detachable and/or interchangeable with a measurement section, for example.

In some exemplary embodiments, at least one section of the flow sensor (e.g., one measurement section thereof), is integrated and/or integrally embodied in the functional device and/or in the fluid circuit, or in at least one section thereof.

In some exemplary embodiments, the flow sensor is arranged and/or embodied to determine at least one volume flow in, at, or on at least two, preferably different, sections of the fluid circuit.

In some exemplary embodiments, at least one flow sensor is arranged and/or embodied in the heat exchange device, and/or it is part of the heat exchange device.

In some exemplary embodiments, the functional device does not comprise any or comprises only one flow sensor. For example, a flow sensor may be part of a further arrangement or device, like a balancing unit of a blood treatment apparatus.

In some exemplary embodiments, the flow sensor or at least one section thereof, is an electromagnetic flow sensor, in particular, a magnetic-inductive flow sensor (hereinafter also referred to shortly as “MID flow sensor”) or it comprises such a MID sensor.

In some exemplary embodiments, at least one flow sensor is a two-channel flow sensor, or comprises a two-channel flow sensor.

In some exemplary embodiments, the functional device and/or a balancing unit is embodied as a tubing system, tubing set, and/or a cassette (e.g., a blood cassette).

In some exemplary embodiments, the functional device and/or the balancing unit is embodied as a single-use article or disposable.

With the expression “single-use article or disposable”, as used herein, it may be understood that the medical functional device is intended for single use, for example in a method for the extracorporeal blood treatment of a patient. It can be provided and/or marketed as a disposable, as single-use article, or the like.

In some exemplary embodiments, the functional device or the balancing unit comprises at least one evaluation unit or evaluation device for determining at least one fluid balance between at least two sections of the fluid circuit, or is connected in data or signal communication with the evaluation unit or evaluation device.

In some exemplary embodiments, the functional device and/or the balancing unit comprises at least one control and/or regulating device (hereinafter denoted for simplification as a “control device,” although it may just as well be a regulating device) for controlling and/or regulating at least one volume flow in at least one section of the fluid circuit. In specific exemplary embodiments, the control device is embodied as a regulating device.

In some exemplary embodiments, the volume flow being controlled by the control device, may be a volume flow of a further section of the fluid circuit (for example the volume flow which flows through a filter device). The volume flow may exemplarily be an ultrafiltration rate.

In some exemplary embodiments, the regulation and/or the control of the volume flow is carried out at least based on the fluid balance and/or at least by evaluating the fluid balance.

In some exemplary embodiments, at least the evaluation device and the control device form a unit.

In some exemplary embodiments, the evaluation device and/or the control device are in data communication with each other and/or with at least one flow sensor, and/or with at least a treatment apparatus, and/or with at least respective sections thereof.

In some exemplary embodiments, the balancing takes place in a medical treatment apparatus (e.g., in a blood treatment apparatus) and/or during a medical treatment (e.g., during a blood treatment). In some exemplary embodiments, this is not the case.

With the expression “at least one volume flow”, it may be herein exemplarily understood as the first and/or the second volume flow, i.e. the volume flow of the first and/or of the second section(s).

In some exemplary embodiments, the fluid balance (also referred to herein in short as FB) may be used as a signal and/or for generating a signal for a control and/or a regulating device for controlling a volume flow of the fluid circuit and/or a pump rate. Controlling the volume flow may include controlling at least one pump of the fluid circuit and/or controlling a cross section of at least one section of the fluid circuit.

In some exemplary embodiments, a fluid balance (FB) may be formed from at least one first volume flow (F1) and one second volume flow (F2). For example, in some exemplary embodiments the fluid balance (FB) may be formed or determined as follows:

FB=F1−F2, or (Formula 1)

FB=F2−F1. (Formula 1′)

In some exemplary embodiments, the fluid balance (FB) is used as a signal and/or for generating a signal for the control device for controlling or regulating a volume flow of the fluid circuit. In some exemplary embodiments, particularly the first or the second volume flow may, for this purpose, be increased or decreased.

A correction may in some exemplary embodiments be directly drawn from electrical evaluation values. For this, the signals which are to be generated out of the received evaluation values are suitably corrected

In certain exemplary embodiments, the control and/or regulating device of the treatment apparatus are in signal communication with the balancing unit. It is configured and/or programmed for executing at least one embodiment of the method, in particular in connection with the respectively required devices.

In some exemplary embodiments, a volume flow is controlled and/or regulated through changing and/or adapting a pump rate of at least one pump of the fluid circuit (or of a connected circuit), or through changing and/or adapting a cross-section of at least one section in the fluid circuit (or of a connected circuit).

In certain exemplary embodiments, the balancing unit is embodied and/or configured to be connectable with at least one flow sensor, comprises at least such a sensor, or is connected to at least such a sensor. The flow sensor may hereby be embodied and/or arranged to measure at least one volume flow in at least the first and/or the second section of the fluid circuit.

In some exemplary embodiments, at least the balancing unit, the functional device, or the treatment apparatus comprises an evaluation unit for forming at least one fluid balance (e.g., a fluid balance between at least two volume flows in different sections of a fluid circuit).

In some exemplary embodiments, the balancing unit, the functional device, or the treatment apparatus comprises at least one evaluation unit for determining at least one fluid balance between at least two sections of the fluid circuit.

In some exemplary embodiments, at least the balancing unit, the functional device, or the treatment apparatus comprises at least one control and/or regulating device for controlling and/or regulating at least one volume flow of the fluid circuit.

In some exemplary embodiments, at least the evaluation device and/or the control device are not embodied or arranged at or on the functional device. For example, the evaluation and/or the control device can be arranged or embodied at or on a treatment apparatus.

In some exemplary embodiments, the medical treatment apparatus is a blood treatment apparatus, in particular a blood cleaning apparatus such as hemofiltration apparatus, a hemodialysis apparatus, a hemodiafiltration apparatus, a general dialysis apparatus, for example an apparatus for acute dialysis, for home dialysis or peritoneal dialysis. It may be an apparatus for executing a method for liver replacement, an apparatus for executing immunoadsorption or the like.

In certain exemplary embodiments, at least one volume flow in a section of the fluid circuit (e.g., a first volume flow in a first section) is adjusted prior to the formation (determination) of the fluid balance.

In some exemplary embodiments, the values of the adjusted or set volume flow, and/or of an adjusted or set and/or recorded temperature, are at least temporarily stored and/or kept.

Some or all of the control and/or regulating devices of the treatment apparatus, the balancing unit, and/or the functional device may respectively comprise devices for executing a method and/or for controlling and/or regulating one or more pumps, such as blood pumps, ultrafiltration pumps, or the like. The respective regulating and/or control devices may alternatively or additionally be at least in signal communication with these devices.

In certain exemplary embodiments, the balancing unit and/or the medical treatment apparatus can be embodied and/or configured to execute the methods described herein.

In some exemplary embodiments, the methods are executed in connection with a functional device, a balancing unit, and/or a blood treatment apparatus.

In some exemplary embodiments, the functional device, the balancing unit, and/or the treatment apparatus does not comprise any scales and/or balance chamber (e.g., any non-ideal, rigid balance chamber).

In certain exemplary embodiments, the method does not comprise gravimetric monitoring, and/or the ultrafiltration rate is not directly measured with a sensor (e.g., not directly measured with a MID sensor).

In some exemplary embodiments, the functional device does not comprise a blood container for the extracorporeal, temporal storage of fluid (e.g., the functional device does not comprise a blood container comprising a cylindrical body).

In some exemplary embodiments, the functional device does not comprise an air separator.

In some exemplary embodiments, the functional device does not comprise a temperature control element which can be integrated into the air separator.

In some exemplary embodiments, the functional device does not comprise a Peltier element.

In some exemplary embodiments, the functional device does not comprise a conveying device or devices (e.g., the functional device does not comprise a pump).

In some exemplary embodiments, the functional device does not comprise a flow-through heater.

In some exemplary embodiments, the functional device does not comprise a temperature control system (e.g., the functional device does not comprise an electronic control for controlling temperature).

Some or all the embodiments described herein may exhibit one, several, or all of the aforementioned and/or the following advantages.

In some embodiments, a precise balancing is advantageously made possible.

This may advantageously take place saving space and costs at the same time.

Measurements made by MID-flow sensors are measurements of a volume flow. The density of the fluid is temperature dependent. Thus, the volume flow as well as the corresponding mass flow are temperature dependent. In some embodiments, however, it is accomplished that the balancing is advantageously achieved independently of the influence of the temperature on the fluid to be balanced.

Furthermore, in certain embodiments, errors resulting from a temperature-dependent, geometrical, and/or volumetric change of the used sensors are avoided.

In some embodiments, temperature-dependent errors (e.g., geometry and/or density errors) are advantageously compensated for (also referred to herein as “corrected”).

In certain embodiments, the errors caused by temperature differences may be reduced by a factor 10 or more, using the methods described herein.

In some exemplary embodiments, measuring the ultrafiltration rate is advantageously avoided. As a result, a sensor to measure the ultrafiltration rate is rendered unnecessary in some embodiments.

In some embodiments, for example in some embodiments of the functional device, of the balancing unit, and/or of the heat exchange device configured as a single-use article, concerns about microbial growth between successive treatments can be alleviated. Furthermore, the risk of infection between patients using the same treatment apparatus may be reduced without involving or requiring particular measures for disinfection.

In certain embodiments, at least one temperature sensor or all temperature sensors may be unnecessary and advantageously dispensed with. Some such methods can equally be executed with one temperature sensor or without any temperature sensor.

In some embodiments, at least one heater or all heaters may be advantageously dispensed with. The methods can equally be executed with one, several, or without any heater.

An advantage of some embodiments is to provide a further balancing unit and a further balancing method for balancing flows of fluid. In addition, a medical functional device as well as a medical treatment apparatus are described herein.

All or some of the advantages achievable by the functional device can be undiminishedly achieved also with the method, and/or with the balancing unit, and/or with the medical treatment apparatus as described herein.

A functional device, which is preferably a medical functional device, is described herein. It comprises, or is connected to, at least one fluid circuit.

In the figures, identical reference numerals denote the same, identical or similar components. The following applies:

### BRIEF DESCRIPTION OF THE FIGURES

FIG. 1 shows, in a simplified illustration, a fluid circuit of a treatment apparatus according to some embodiments described herein;

FIG. 2 shows, in a highly simplified illustration, a first exemplary functional device according to some embodiments described herein having two flow sensors;

FIG. 3 shows, in a highly simplified illustration, an exemplary arrangement of a treatment apparatus having a further functional device according to some embodiments described herein;

FIG. 4 shows, in a highly simplified illustration, a further functional device according to some embodiments described herein;

FIG. 5 shows, in a highly simplified illustration, a further functional device according to some embodiments described herein; and

FIG. 6 shows, in a highly simplified illustration, yet a further functional device according to some embodiments described herein.

### DETAILED DESCRIPTION OF ILLUSTRATIVE EMBODIMENTS

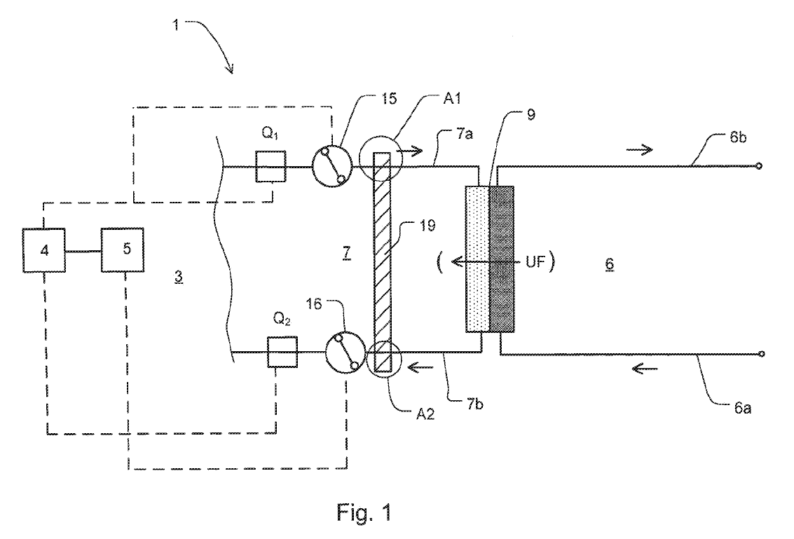

FIG. 1 shows, in a highly simplified illustration, a section of a fluid circuit 1 which is connected in fluid communication with, or which is a part of, a treatment apparatus 3 according to some embodiments described herein. The treatment apparatus 3 is connected to a balancing unit 4, or the treatment apparatus 3 comprises the balancing unit 4. The treatment apparatus 3 or the balancing unit 4 is/are in addition connected to or comprise(s) a control or regulating device 5 for controlling or regulating pumps, valves, etc. for performing the treatment of the patient. The balancing unit 4 is further connected to two flow sensors Q1, Q2 or is connected to at least one two-channel flow sensor, or comprises the latter. In the example shown, the flow sensors are not illustrated as part of the treatment apparatus 3. Alternatively, both or at least one flow sensor Q1 and/or Q2 may be designed as part of the treatment apparatus 3 or may be arranged in it or attached to it.

The balancing unit 4 may optionally comprise the control or regulating device 5, or may be in signal communication with it, as exemplarily shown in FIG. 1.

The treatment device 3 illustrated in FIG. 1, and in all the following figures, is purely exemplarily designed as an ultrafiltration device; the fluid circuit 1 as a circuit for the extracorporeal blood treatment. Both of these assumptions serve solely for the better understanding of the inventive concepts described herein with respect to concrete examples, and should in no way limit the inventive concepts.

The fluid circuit 1 comprises an extracorporeal blood circuit 6 and a dialysate circuit 7, which, purely exemplarily, are in both substance exchange and fluid exchange by means of a filter or filtering device 9. The arrows denote a flow direction of different fluids during a treatment of a patient (not illustrated) by the treatment apparatus 3.

Blood of the patient flows into the extracorporeal blood circuit 6 through an arterial blood line 6a to the filter device 9 to be cleaned. The cleaned blood is returned to the patient through a venous blood line 6b. The blood lines 6a, 6b may comprise corresponding hose clamps.

In some embodiments, the dialysate circuit 7 (which may be a substituate or dialysate circuit, and which comprises a dialysis fluid line 7a and a dialysate line 7b, a first pump 15, and a second pump 16) may for simplification be disposed or arranged for generating a particular volume flow within the dialysate circuit 7. The pumps 15, 16 are, as suggested by the dashed line, optionally each in signal communication with the control or regulating device 5.

The dialysate circuit 7 comprises further a heat exchange device 19 arranged between a first section A1 and a second section A2 of the fluid circuit 1, and which allows and causes a heat balance or compensation between both sections A1 and A2.

Additionally, the blood circuit 6 may optionally comprise at least one pump (not illustrated in the figures) for conveying blood. Such a pump may also be in signal communication with the control or regulating device 5.

In FIG. 1, an arrow indicates an ultrafiltration, occurring optionally, in which fluid from the blood circuit 6 enters with a flow or volume flow UF (ultrafiltration) through the filter 9 into the dialysate circuit 7. The ultrafiltration rate UF may be set, adjusted, or controlled and/or regulated by means of the control or regulating device 5 and respective pumps (e.g., the first pump 15, the second pump 16, and/or a blood pump, not illustrated).

The arrangement of FIG. 1 is suitable to execute the methods described herein due to the programmed balancing unit 4 and/or to the programmed control or regulating device 5.

The pumps 15, 16, illustrated in FIG. 1 and at least the non-illustrated blood pump will not be repeatedly illustrated in the following figures, for the sake of simplicity. They may however be optionally provided unchanged in the following described embodiments.

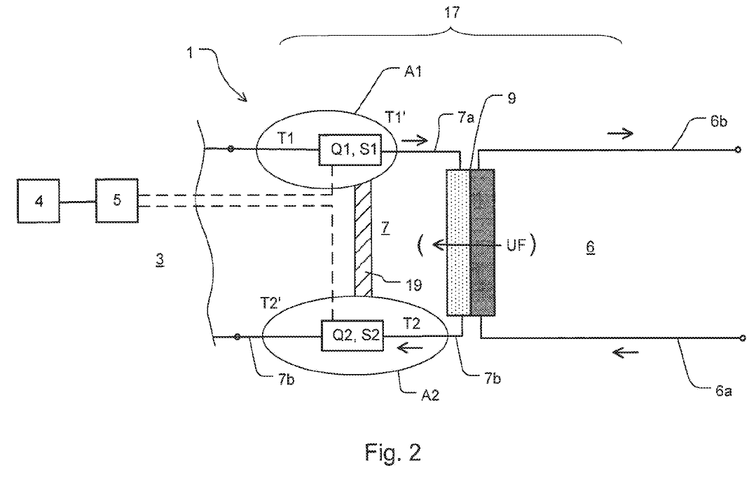

FIG. 2 shows, in a highly simplified illustration, a functional device 17, which comprises parts of the dialysate circuit 7 and, purely optionally, also parts of the blood circuit 6. While the functional device 17 does not usually comprise the filter 9, it may however be connected in fluid communication therewith.

A first section A1 of the dialysate circuit 7 comprises or is connected to a first flow sensor Q1. A second section A2 of the dialysate circuit 7 comprises or is connected to a second flow sensor Q2. The first and/or the second flow sensor Q1, Q2 is/are optionally part of the functional device 17.

The first section A1 is exemplarily part of the dialysis fluid line 7a, and the second section A2 is exemplarily part of the dialysate line 7b.

The temperature of the fluid flowing out from the first section A1 (the first section A1 being disposed upstream of the filter device 9 in the depicted embodiment) shall be denoted as T1′.

The temperature of the fluid entering or flowing into the first section A1 shall be denoted as T1.

The temperature of the fluid which flows out from the second section A2 shall be denoted as T2′. The second section A2 is disposed downstream of the filter device 9 in the depicted embodiment.

The temperature of the fluid that enters or flows into the second section A2 shall be denoted as T2.

The temperature T1 may be determined by an optional temperature sensor S1. The temperature sensor S1 is however not required, in particular when the temperature T1 is known or can be estimated. For example, in some treatment scenarios, it can be assumed in sufficiently good approximation, that T1 (the temperature with which the fluid flows into the first section A1) corresponds to the temperature the fluid has in a fluid source.

The temperature T2 may be determined by an also optional temperature sensor S2. This temperature sensor S2 is however not required, in particular when the temperature T2 is known or can be estimated. In fact, used dialysate generally exhibits a low temperature variation when flowing out of the filter device 9. Generally, the temperature of the fluid being discharged from the filter device 9 varies within the range of the body temperature (ca. 37° C.). It may therefore be assumed, in some treatment scenarios, in sufficiently good approximation, that T2 corresponds to the body temperature of the patient.

As suggested in FIG. 2 by a dashed line, the sensors Q1, Q2 and, if applicable S1 and/or S2, are in signal communication with the control or regulating device 5, the evaluation device, and/or the balancing unit 4.

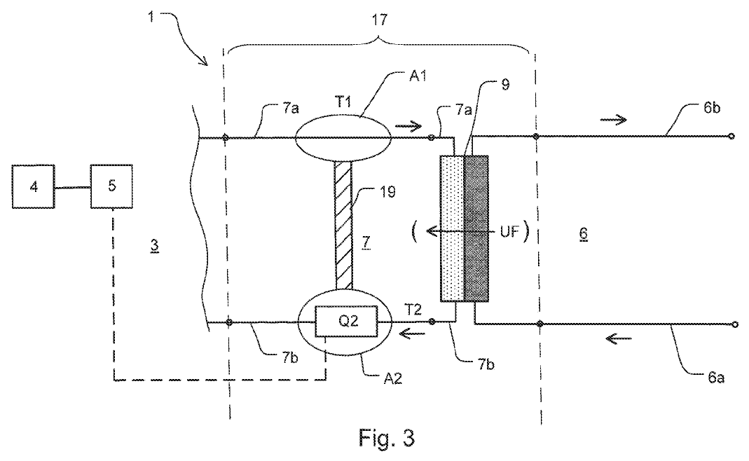

FIG. 3 shows, in a highly simplified illustration, a functional device 17 in a further exemplary embodiment in accordance with some embodiments described herein. The functional device 17 corresponds substantially to that of FIG. 2.

FIG. 3 also shows that the first flow sensor Q1 may be unnecessary in some embodiments. This can be the case, for example, when the first flow F1 is known or can be estimated, or the second volume flow F2 is adjustable and/or set by a device suitable for this purpose (e.g. a pump and/or a control or regulating device and/or an input device).

The particular feature of the embodiment of FIG. 3, according to which there is no necessity of a flow sensor Q, and/or of a temperature sensor S1 for the first section, and/or a temperature sensor S2 for the second section A2, is combinable, where useful, with any other embodiment described herein.

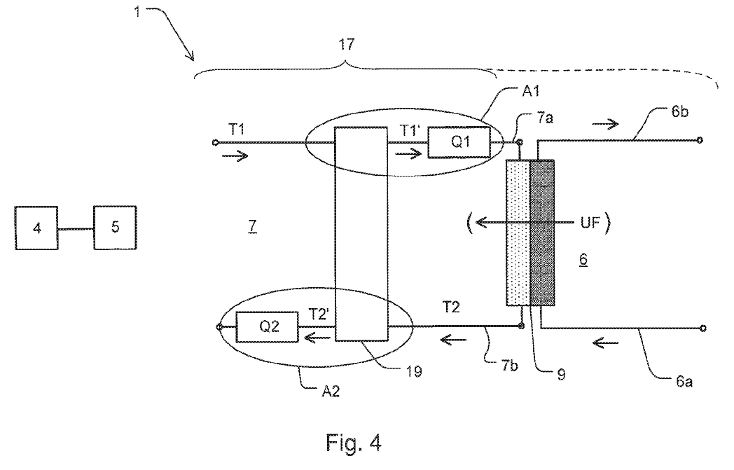

FIG. 4 shows in a highly simplified illustration a further embodiment of the functional device 17.

The functional device 17 comprises, optionally, a first and second flow sensor Q1 and Q2. The functional device 17 comprises or is connected to at least one heat exchange device 19.

The optionally provided first sensor Q1 is arranged in a first section A1 of the substituate or dialysate circuit 7 of the fluid circuit 1, preferably in the dialysis fluid line 7a. The optionally provided second flow sensor Q2 is arranged in a second section A2 of the substituate or dialysate circuit 7 of the fluid circuit 1, preferably in the dialysate line 7b.

The first section A1 is optionally positioned upstream of a filter device 9 (for example a blood filter), the second section A2 is optionally positioned downstream of the filter device 9. In this example, the first and the second sections A1, A2 are optionally each in fluid communication with the filter device 9.

The heat exchange device 19 is arranged with regard to the flow direction of the fluid during a blood treatment (said direction being indicated by arrows) for an exchange of heat or thermal energy between the fluid flowing through the first section Al and the fluid flowing through the second section A2.

In particular, the heat exchange device 19 is optionally arranged upstream of the first flow sensor Q1, which is itself arranged upstream of the filter device 9. The heat exchange device 19 is at the same time optionally arranged upstream of the second flow sensor Q2, which is itself arranged downstream of the filter device 9.

The heat exchange device 19 may optionally be part of the first section A1 and/or of the second section A2 (e.g., an integral part thereof), and/or it may optionally only be connected (e.g., thermally) with the first section A1 and/or with the second section A2.

The heat exchange device 19 is provided, suitable, and/or designed to effect a heat exchange between the fluid flowing through the first section A1 and the fluid flowing through the second section A2.

A heat balance as used herein may mean that the difference between: (i) a temperature T1′ of the fluid after leaving the heat exchange device 19 but before or at the first flow sensor Q1, and in particular before the filter device 9, and (ii) a temperature T2′ of the fluid flowing out after passing the heat exchange device 19 but before or at the second flow sensor Q2, is zero or substantially zero. It may also be understood, that the above-mentioned difference is in any case less than the difference between the temperature T1 and T2, which the fluid or fluids respectively had before entering in the heat exchange device 19.

In some embodiments, one or more devices are included to detect the temperature T1 and/or the temperature T2 (e.g., by at least one temperature sensor, not illustrated), and/or to set or adjust the temperature T1 and/or the temperature T2 (e.g., by a suitable device such as a heater).

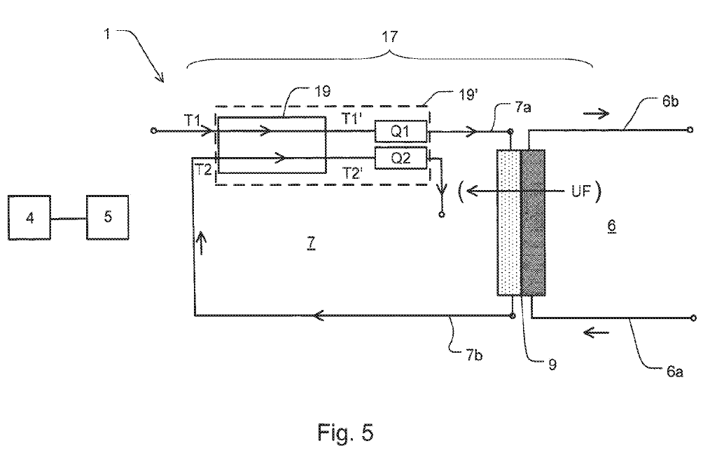

FIG. 5 shows, in a highly simplified illustration, yet another further exemplary embodiment of the functional device.

The arrangement of the heat exchange device 19 and the flow sensors Q1 and Q2 corresponds basically to that of the FIG. 4. The flow sensors Q1 and Q2 of the heat exchange device 19 are each arranged downstream with respect to a flow direction of the fluid (of a medical fluid).

Likewise, the heat exchange device 19 is provided and/or arranged to effect a heat balance between the sections that are each arranged downstream, in particular directly, after the heat exchange device 19.

The section denoted with the reference numeral 19′ may be designed as a removable or detachable section, or as a sector or insert, which may be removed from or out of the functional device 17. This removable section 19′ comprises at least the heat exchange device 19. In addition, this removable section 19′ may optionally comprise one or both flow sensors Q1, Q2, or at least sections thereof, as exemplarily illustrated.

The removable section 19′ may optionally be designed as a single-use item or as a disposable. The same may apply to the functional device 17 in some embodiments.

The removable section 19′ may optionally comprise at least one solid body, in particular a highly thermal or heat conductive body, for example a metallic hard part, in which the respective sections A1, A2 of the fluid circuit 7 extend.

Both respective sections A1, A2 of the fluid circuit 7 may exemplarily be separated from each other within the heat exchange device 19 through a thin, in particular fluid-tight, film and/or plate.

Additionally, in this case, it may optionally be intended to use only one flow sensor Q1 or Q2, in particular when at least one of the two flows F1 and/or F2, with which the fluid flows through the sections A1, A2, respectively, is already set and/or is adjustable.

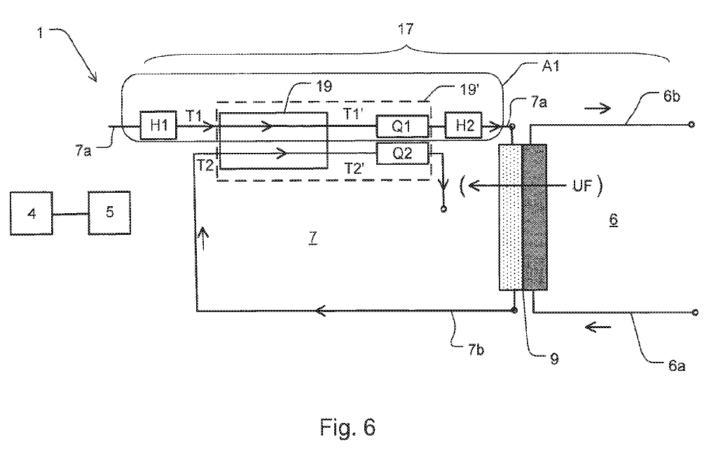

FIG. 6 shows in a highly simplified illustration the functional device 17 in a yet further embodiment. Thereby, the arrangement and/or the design of the removable section 19′ of FIG. 5 or another section of the functional device 17 may additionally comprise at least one heater Hi (i=1, 2).

The heater Hi may be arranged particularly in the first section A1 of the fluid circuit 1.

There are two heaters H1 and H2 illustrated in FIG. 6. Both are exemplarily arranged and/or designed in the first section A1. The first heater H1 is arranged upstream or before the removable section 19′, while the second heater H2 is arranged downstream or after the removable section 19′. Both heaters H1, H2 are, not part of the functional device 17. In some embodiments, they may however be part of the functional device 17. Both flow sensors Q1, Q2 are not part of the functional device 17. They may be part of the balancing unit 4 and/or of the treatment apparatus 3.

In some embodiments, only a single heater H1 or H2 is included in or along the fluid circuit 1.

In some embodiments, at least one heater Hi (e.g., in the first section A1 of the fluid circuit 1) is arranged upstream of the heat exchange device 19 and/or downstream of a flow sensor (not illustrated) which is arranged downstream of the heat exchange device 19.

In a further, not illustrated embodiment of the functional device 17, a heater Hi may optionally be designed and/or arranged at, in, and/or at least in connection with the heat exchange device 19, the removable section 19′, or at least sections thereof, respectively.

The heater Hi may be designed to guarantee or ensure a predetermined minimum temperature of the fluid. Thus, a measurement of temperature may be unnecessary in certain embodiments.

Heaters Hi, as shown in FIG. 6, may be included in the arrangements of any of the embodiments shown in the preceding figures.

By using the arrangements of the figures and the particular configuration of the balancing unit 4, a correct fluid balance can be generated. Alternatively or additionally, an ultrafiltration rate may be adapted or adjusted by the control and/or regulating device 5.

### LIST OF REFERENCE NUMERALS

1 fluid circuit
3 blood treatment apparatus
4 balancing unit
5 control or regulating device
6 blood circuit
6a arterial blood line
6b venous blood line
7 dialysate and/or substituate circuit
7a dialysis fluid line
7b dialysate line
9 filter or filtering device
15 first pump
16 second pump
17 functional device
19 heat exchange device
19′ removable section of the functional device
A1 first section of the dialysate and/or substituate circuit
A2 second section of the dialysate and/or substituate circuit
Q1 first flow sensor
Q2 second flow sensor
S1 temperature sensor
S2 temperature sensor
F1 first volumetric flow in the first section of the fluid circuit
F2 second volumetric flow into the second section of the fluid circuit
Hi (i=1, 2, . . . ) heater

1.-16. (canceled)

17. A medical device, comprising a fluid circuit or one section thereof, wherein the medical device or the fluid circuit comprises a heat exchange device or is connected thereto, wherein the heat exchange device is arranged between a first section of the fluid circuit and a second section of the fluid circuit.

18. The medical device according to claim 17, wherein the heat exchange device is configured to balance a temperature of fluid in the first section and a temperature of fluid in the second section, or the heat exchange device is configured such that a temperature difference of the fluids in the first and second sections is at least reduced before and after exiting the heat exchange device.

19. The medical device according to claim 17, wherein the heat exchange device comprises a heat conducting body, and wherein a section of the fluid circuit extends along or is arranged in the heat conducting body.

20. The medical device according to claim 17, wherein the heat exchange device is a separate, substitutable, or interchangeable device which is releasable from the medical device as a disposable item.

21. The medical device according to claim 17, wherein the medical device is configured to be connected or connectable to a flow sensor or comprises a flow sensor, wherein the flow sensor is arranged to measure a volume flow in either the first section or the second section, or in both of the first section and the second section of the fluid circuit.

22. The medical device according to claim 21, wherein the flow sensor is a magneto-inductive flow sensor, or is a section thereof.

23. The medical device according to claim 21, wherein the flow sensor is a two-channel flow sensor.

24. The medical device according to claim 21, wherein the flow sensor is arranged in the heat exchange device or is part thereof.

25. The medical device according to claim 17, wherein the medical device comprises a tubing system, or a cassette, or both.

26. The medical device, according to claim 17, wherein the medical device is a single-use item or is disposable.

27. A balancing unit for determining a fluid balance between a first volume flow in a first section of a fluid circuit and a second volume flow in a second section of the fluid circuit, wherein the balancing unit comprises a medical device or is at least in data communication with a medical device or with parts thereof, wherein the medical device comprises a fluid circuit or one section thereof, wherein the medical device or the fluid circuit comprises a heat exchange device or is connected thereto, wherein the heat exchange device is arranged between a first section of the fluid circuit and a second section of the fluid circuit.

28. The balancing unit according to claim 27, wherein the balancing unit is configured to be connected or connectable with a flow sensor or comprises a flow sensor, wherein the flow sensor is arranged to measure a volume flow in either the first or the second section, or in both of the first section and the second section of the fluid circuit.

29. The balancing unit according to claim 28, wherein the balancing unit: (i) further comprises an evaluation unit for generating a fluid balance between a first volumetric flow in the first section of the fluid circuit and a second volumetric flow in the second section of the fluid circuit, or (ii) further comprises a control device for controlling at least one further volume flow of the fluid circuit, or (iii) further comprises both the evaluation unit of (i) and the control device of (ii).

30. The balancing unit of claim 29, wherein the evaluation unit or the control device is part of a treatment apparatus, or is arranged at or in a treatment apparatus.

31. A medical treatment apparatus for treating a medical fluid, the medical treatment apparatus having a balancing unit, or having a medical device, or having both a balancing unit and a medical device, wherein the balancing unit is configured for determining a fluid balance between a first volume flow in a first section of a fluid circuit and a second volume flow in a second section of the fluid circuit, wherein the medical device comprises a fluid circuit or one section thereof, wherein the medical device or the fluid circuit comprises a heat exchange device or is connected thereto, wherein the heat exchange device is arranged between the first section of the fluid circuit and the second section of the fluid circuit.

32. A method for controlling a volume flow in a section of a fluid circuit, the method comprising:
providing: (i) a medical device comprising the fluid circuit or one section thereof, wherein the medical device or the fluid circuit comprises a heat exchange device or is connected thereto, wherein the heat exchange device is arranged between a first section of the fluid circuit and a second section of the fluid circuit, or (ii) a treatment apparatus comprising a balancing unit configured for determining a fluid balance between a first volume flow in a first section of the fluid circuit and a second volume flow in a second section of the fluid circuit, or (iii) both the medical device of (i) and the treatment apparatus of (ii);
determining or setting the first volume flow in the first section of the fluid circuit;
determining the second volume flow in the second section of the fluid circuit;
determining the fluid balance between the first volume flow and the second volume flow; and
using the determined fluid balance for controlling a volume flow of the fluid circuit, a pumping rate of a pump of the fluid circuit, or a cross-section of a section of the fluid circuit.
